(19) United States
(12) Patent Application Publication
(10) Pub. No.: US 2009/0318557 A1
(43) Pub. Date: Dec. 24, 2009

(54) Dermatological compositions

(76) Inventor: Richard F. Stockel, Bridgewater, NJ (US)

Correspondence Address: Law Offices of Jack Matalon, 32 Shelley Rd., Springfield, NJ, 07081-2529, US

(21) Appl. No.: 12/583,919

(22) Filed: Aug. 27, 2009

Related U.S. Application Data
(63) Continuation-in-part of application No. 11/637,450, filed on Dec. 12, 2006.
Application No. 11/637,450 is a continuation-in-part of application No. 10/741,346, filed on Dec. 22, 2003, now abandoned.
(60) Provisional application No. 61/196,455, filed on Oct. 17, 2008.

(51) Int. Cl. A61K 31/195; A61K 31/19

(52) U.S. Cl. 514/565; 514/568

(57) ABSTRACT

A dermatological composition comprising a salt of a cation and an anion. The cation is derived from a monomeric or polymeric molecule that will generate an amidine moieity, a guanidine moieity or a biguanide moieity. The anion is derived from a monomeric or polymeric molecule that will generate a carboxylic acid moieity. The composition may be prepared by a metathesis or acid-base reaction.

### REFERENCE TO RELATED APPLICATIONS

This application is a continuation-in-part of application Ser. No. 11/637,450 filed Dec. 12, 2006 which in turn was filed as a continuation-in-part of application Ser. No. 10/741,346 filed Dec. 22, 2003 (now abandoned). This application is also a continuation-in-part of application Ser. No. 61/196,455 filed Oct. 17, 2008. The disclosures of the foregoing applications are hereby incorporated herein in their entirety.

### FIELD OF THE INVENTION

The invention relates to the dermatological compositions containing bioactive salts providing exceptional antimicrobial, antibacterial, antiviral and/or antifungal activity and reduced undesirable side effects such as skin irritation and can be used for the treatment of mammalian skin, hair and nail disorders. The unique bioactive salts of the invention have been found to be extremely useful for the treatment of many skin conditions such as, but not limited to, ichthyosis, eczema, dry skin psoriasis, pruritis, palmar, plantar hyperkeratosis, acne, keratoses, herpes virus, skin blemishes, warts, etc.

The invention provides compositions and methods for the alleviation of both visible and non-visible, i.e., pre-emergent, dermatological lesions associated with changes in normal keratinization, cutaneous infection, epidermal formation or pilosebaceous function such as acne, psoriasis, seborrhea, ingrown hairs and pseudofolliculitis barbae, and hyper-pigmented skin.

The invention significantly expands the options for treatment of dermatological conditions by allowing the medical professional to choose from a plethora of bioactive agents having the proper chemical characteristics required to prepare a composition to be used for a specific skin condition. For example a cationic (or conjugate base) molecule with antimicrobial, antibacterial, antiviral or antifungal properties can be combined with a selected anionic (or conjugate acid, respectively) molecule to provide the desired therapeutic outcome as well as a benefit to the skin.

The use of environmentally-beneficial materials, especially those that are referred to as “green” is an important consideration in selecting a topically-applied or ingested composition. The use of natural or naturally-derived materials is also of significant interest for topically-applied or ingested compositions. Accordingly, the use of both green and naturally-derived materials in a composition having exceptional antimicrobial, antibacterial, antiviral and/or skin-beneficial properties would be clearly desirable. If all of the components of the compositions of the invention are GRAS (Generally Regarded As Safe) and are approved for food use, the resulting compositions could also be ingested with little or no side effects.

In respect to the term “dermatological” used throughout the specification and claims, it should be understood that the compositions of the invention are useful not only for the treatment of mammalian skin, but also for the treatment of mammalian hair and the treatment of mammalian nails. Furthermore, it should be understood that the compositions of the invention may be applied not only topically to mammalian skin, hair or nails, but may also be ingested where a particular mammalian skin, hair or nail condition so dictates.

### BACKGROUND OF THE INVENTION

The prior art is replete with various approaches to the treatment of dermatological conditions. Benzoyl peroxide is well known as a medicament for the treatment of acne. Often the benzoyl peroxide is combined with an antibacterial agent or an antibiotic to extend its spectrum of activity as disclosed in, e.g., U.S. Pat. No. 5,767,098. However there are several disadvantages to this combination approach. With prolonged usage that is typically required for the treatment of acne, the bacterial flora become resistant thus rendering the antibacterial agent or the antibiotic less effective in subsequent treatment. Moreover, the benzoyl peroxide component of the combination is oxidatively unstable.

Numerous publications and patents disclose the use of α-hydroxy acids for the treatment of dermatological conditions. For example, U.S. Pat. No. 4,363,815 discloses the use of such compounds for the treatment of dry skin, ichthyosis, plantar hyperkeratosis, Darier's disease, keratoses, acne, psoriasis, eczema, pruritis, warts and herpes virus. Other patents describe the use of various incipients to lessen skin irritation and stinging, e.g., lactate salts, amphoteric salts (see U.S. Pat. No. 5,420,106), ascorbic acid derivatives (U.S. Pat. No. 5,703,122), amino salts (see Cosmetics and Toiletries, volume 113, March 1998, p. 55).

Salicyclic acid is frequently disclosed as an active ingredient for the treatment of a wide variety of skin conditions, e.g., psoriasis, skin atrophy, skin wrinkles, acne, etc., see U.S. Pat. Nos. 5,776,920; 5,780,457; 5,780,458; 6,436,417. In all of these cases, salicyclic acid must be modified with other incipients to prevent undesirable side reactions such as skin irritation and the like.

There is a need for a safe and efficacious course of treatment for severe acne. The only treatment to date which has proven to be uniformly effective is isotretinoin which is orally administered. This medication has many undesirable side effects including the possibility of causing birth defects when administered to pregnant women.

If a composition included GRAS ingredients that have an antimicrobial component as well as a naturally-occurring component that provides skin benefits, then such approach would be beneficial in reducing potential irritation from harsh chemicals while improving skin health. The component in the composition that provides skin benefits could include, e.g., vitamin C and its derivatives (salts or esters) such as palmitates, phosphates such as magnesium ascorbyl phosphate and sodium ascorbyl phosphate, and tetra-substituted liphophilic ascorbates, etc.; vitamin A and its derivatives such as palmitates, etc.; essential fatty acids, e.g., omega acids including 3,6,9 types, etc.; alpha-hydroxy acids and their derivatives (esters and salts), e.g., alpha-hydroxyacetic acid (also known as glycolic acid), alpha-hydroxypropionic acid (also known as lactic acid), alpha-hydroxytetranoic acid, alpha-hydroxyhexanoic acid, alpha-hydroxyoctanoic acid (also known as alpha-hydroxy-caprylic acid), alpha-hydroxynonanoic acid, alpha-hydroxydecanoic acid, alpha-hydroxy-undacanoic acid, alpha-hydroxydodecanoic acid (also known as alpha-hydroxylauric acid), alpha-hydroxytetradecanoic acid, alpha-hydroxyhexadecanoic acid, alpha-hydroxy-octadecanoic acid, alpha-hydroxyoctaeicosanoic acid, etc.; beta-hydroxy acids and their derivatives (esters and salts), e.g., salicylic acid, etc. Any hydroxy acid that alleviates the symptoms of an undesirable skin condition may be used. Accordingly, the hydroxyl acid may be an alpha, beta, gamma, delta, epsilon or omega hydroxyl acid. If the antimicrobial component of the composition is considered “Green and Naturally Derived”, the resultant composition would be preferable to those compositions that utilize natural materials to treat skin conditions.

### SUMMARY OF THE INVENTION

The invention pertains to a dermatological composition comprising a salt of a monomeric or polymeric cation and a monomeric or polymeric anion. The cation may be an amidine moieity, a guanidine moieity or a biguanide moieity, while the anion may be a carboxylic acid moieity. Such compositions will possess antimicrobial, antibacterial, antifungal, antiviral and/or mammalian skin, hair and/or nail-beneficial properties.

### The Salts of the Invention

The salts of the invention employed in the dermatological compositions of the invention may be formed by a metathesis or acid-base reaction. In the case of either type of reaction, a monomeric or polymeric cationic molecule is reacted with a monomeric or polymeric anionic molecule. For the metathesis reaction, the cationic molecule is chosen such that the resultant salt will contain a cationic moieity that may be an amidine, a guanidine or a biguanide and the anionic molecule is chosen such that the salt will contain a carboxylic acid anionic moieity. For the acid-base reaction, the cationic molecule will be present in the form of a free base such that the resultant salt will contain a cationic moieity that may be an amidine, a guanidine or a biguanide and the anionic molecule will be a carboxylic acid capable of protonating the free base thereby resulting in a salt that will contain a carboxylic acid anionic moieity. The features of the metathesis and the acid-base reactions are discussed hereinbelow. For the purposes of the present invention, the preferred reaction is the metathesis reaction.

As mentioned above, the monomeric or polymeric cationic molecules include guanidines, amidines and bigauanides. Such materials possess superior antimicrobial activity and may be characterized as having two or three nitrogen atoms attached to a carbon atom that will readily accept a proton to form a protonated imino functionality. The driving force for this to occur is due to resonance in forming an energetically stabilized protonated imino group in the electronic ground state. Furthermore, each of the functionalities from a monoacid base with pKₐ's from about 7.5 to about 13, because by accepting a proton, they form a symmetrical cation that is stabilized by delocalization. Such antimicrobial compounds may be monomeric or polymeric and may contain functional groups such as aliphatic, aromatic or alicyclic groups. The cidal mechanism for all of these functionalities is the same.

Suitable examples of cationic molecules that will result in a cationic amidine moieity are propamidine and dibromopropamidine. Suitable examples of cationic molecules that will result in a cationic biguanide moieity are chlorhexidine, hexetidine, alexidine and polyhexamethylene biguanide hydrochloride.

Preferably, the cationic molecule that will result in a cationic guanidine moieity comprises a N^{α}—(C₁-C₂₂) alkanoyl di-basic amino acid (C₁-C₂₂) alkyl ester such as N′-lauroyl-L-arginine ethyl ester which is typically employed as the hydrochloride salt (N^{α}-lauroyl-L-arginine ethyl ester is also referred to hereinbelow as “LAE”). Other suitable examples of cationic molecules that will result in a cationic guanidine moieity are N^{α}-lauroyl-L-histidine ethyl ester and N^{α}-lauroyl-L-tryptophan ethyl ester.

As mentioned above, the anionic molecule is chosen such that the salt will contain a monomeric or polymeric carboxylic acid moieity. Such carboxylic acid moieities may be obtained from monomeric or polymeric, saturated or unsaturated carboxylic acids containing functional groups such as aliphatic, aromatic or alicyclic groups. Typically, the anionic molecule is one which will result in a monobasic aliphatic acid moieity or a monobasic aromatic acid moieity. Preferably, the monobasic aliphatic carboxylic acid moieity is derived from a monobasic aliphatic carboxylic acid molecule such as lauric acid, palmitic acid, myristic acid, oleic acid, stearic acid, dehydroacetic acid and undecylenic acid. Alternatively, the monobasic aromatic carboxylic acid moieity may be derived from a monobasic aromatic carboxylic acid molecule that contains a phenol group such as mandelic acid or salicylic acid (which is preferred).

For many dermatological applications, it is preferred that the monobasic aliphatic or aromatic carboxylic acids contain a hydroxy group, e.g. an alpha-hydroxy group or a beta-hydroxy group or a ketone group. Monobasic aliphatic carboxylic acids containing an alpha-hydroxy group are most preferred. Suitable examples of such carboxylic acids are glycolic acid, gluconic acid, glyceric acid and lactic acid.

The anionic molecule may also be one that will result in an aliphatic or aromatic carboxylic acid moieity in which at least two carboxylic acid groups will be present. Suitable examples of such anionic molecules are citric acid, malic acid, tartaric acid and azelaic acid.

It is preferred that the cationic and anionic molecules be chosen such that the resultant salt will exhibit a maximum solubility in aqueous media of about 5 wt. %, preferably 2 wt. %. Such salts have been found to be useful for those dermatological applications where prolonged extended-release of the salt is desirable. The extended-release and also possible increased substantivity of such low water-solubility salts appear to be due to the charged nature of the species and the slow dissociation of the salts after application to a surface, e.g., the skin, hair or nails.

Especially preferred salts of the invention are the laurate of N^{α}-lauroyl-L-arginine ethyl ester, the salicylate of N^{α}-lauroyl-L-arginine ethyl ester, the lactate of N^{α}-lauroyl-L-arginine ethyl ester, the citrate of N^{α}-lauroyl-L-arginine ethyl ester, the malate of N^{α}-lauroyl-L-arginine ethyl ester, the gluconate of N^{α}-lauroyl-L-arginine ethyl ester, the azelate of N^{α}-lauroyl-L-arginine ethyl ester, the glycolate of N^{α}-lauroyl-L-arginine ethyl ester, the glycerate of N^{α}-lauroyl-L-arginine ethyl ester, the hyaluronate of N^{α}-lauroyl-L-arginine ethyl ester, the arachidonate of N^{α}-lauroyl-L-arginine ethyl ester, the oleate of N′-lauroyl-L-arginine ethyl ester (C₁₈, unsaturated), the linoleate of N^{α}-lauroyl-L-arginine ethyl ester (C₁₈, polyunsaturated), the α-linoleate of N^{α}-lauroyl-L-arginine ethyl ester acid (ALA), the eicosapentaenoate of N^{α}-lauroyl-L-arginine ethyl ester acid (EPA), the docosahexaeonate of N^{α}-lauroyl-L-arginine ethyl ester (DHA), the erucate of N^{α}-lauroyl-L-arginine ethyl ester, the tartrate of N^{α}-lauroyl-L-arginine ethyl ester and the 3-hydroxypropionate of N-lauroyl-L-arginine ethyl ester.

The salts of the invention may be prepared either prior to inclusion into a specific dermatological composition, but also as an in-situ reaction while preparing the dermatological composition For many, but most certainly not all, applications, the salts of the invention will be contained in compositions in the form of emulsions, nano-emulsions, micro-emulsions, gels, creams, dispersions, suspensions, foams, sprays, etc. Where a particular skin/hair/nails condition so dictates, the salts of the invention may be orally administered in the form of tablets or capsules by compounding the salts with the usual excipients well-known in the pharmaceutical field.

The salts of the invention have been found to be extremely effective against a wide variety of microorganisms, e.g., bacteria and fungi. Moreover, the salts have important safety, efficacy and toxicity implications since the cationic and anionic molecules employed in the preparation of the salts by either a metathesis or an acid-base reaction are typically those that have been approved for use by the EPA or the FDA.

### Formation of Emulsions of the Salts of the Invention

As mentioned above, the salts of the invention have limited water solubility. Therefore, for many dermatological applications, it is desirable to utilize the salts in the form of emulsions, nano-emulsions or micro-emulsions. The following is a generalized procedure for preparing emulsions, nano-emulsions or micro-emulsions of the salts.

First, the salt of the invention is dissolved in the minimum amount of a solvent that will completely dissolve the selected salt in the amount that is intended for use in the desired dermatological medicament. The solvent of choice will be one with the appropriate Hildebrand solubility parameter. The solubility parameter is a numerical value that indicates the relative solvency behavior of a specific solvent. Hildebrand solubility parameters of about 8.5 to about 22.0 are generally suitable for solubilization of the salts. Exemplary solvents with the requisite Hildebrand solubility parameters include ethanol, glycerin, propylene glycol, sorbitol, methanol and the like.

The desirable Hildebrand solubility parameter will depend on the ionic/covalent bonding energies of the salts of the invention. The correct solvent will be one having a relatively low Hildebrand solubility parameter if the bonding has more covalency and a relatively high Hildebrand solubility parameter if the bonding is more ionic. Of course, combinations of correct solvents may also be utilized to dissolve the salts of the invention.

Thereafter, a surfactant is added to the dissolved salt. The surfactant may be cationic, anionic or amphoteric in nature, and combinations of the different types or combinations of the same type of surfactants may be used. Preferably, the surfactant will be amphoteric or nonionic in nature. Highly negative anionic surfactants are not very functional.

The last step is to dilute the salt-solvent-surfactant composition with water to the concentration desired for the selected dermatological medicament so as to form an emulsion, nano-emulsion or micro-emulsion depending on the micellar size and the choice of solvents/cosolvents.

### The Surfactants

For the purposes of this invention, it is preferred that the surfactants employed in the formation of emulsions, nano-emulsions or micro-emulsions of the salts of the invention are generally of the nonionic or amphoteric type or combinations of one or more nonionics, one or more amphoterics or one or more nonionics in combination with one or more amphoterics. Also, a cationic-amphoteric or cationic-nonionic surfactant system can be utilized to provide satisfactory results. Highly charged anionic surfactants are less desirable since they have the potential to reduce the bioactivity of the salts by causing some degree of precipitation, thereby lessening the effectiveness of the salts.

It has also been found that cationic phospholipids, preferably in combination with nonionic and/or amphoteric surfactants are effective in the formation of micro-emulsions or emulsions of the salts of the invention.

Surfactants that carry a positive charge in strongly acidic media carry a negative charge in strongly basic media, and form zwitterionic species at intermediate pH levels are amphoteric. The preferred pH range for stability and effectiveness is about 5.0 to about 9.0. Within this pH range, the amphoteric surfactant is mostly or fully in the zwitter (neutral) form, thereby negating any dilution of bioactivity of the salts of the invention, provided that the surfactant is employed in the preferred concentration range of about 0.25 to about 6.0 wt. %, based on the weight of the salt of the invention in the final formulation.

The following surfactants have been found to be effective in the formation of emulsions, nano-emulsions and micro-emulsions of the salts of the invention: amphoteric amidobetaines; nonionic polyethoxylated sorbitol esters, polycondensates of ethylene oxide-propylene oxides (polyoxamers), polyethoxylated hydrogenated castor oils, and certain cationic phospholipids.

Suitable examples of amidobetaines include cocoamidoethyl betaine, cocoamido-propyl betaine; and mixtures thereof. Other suitable amphoteric surfactants include long chain imidazole derivatives such as the product marketed under the trade name “Miranol C2M” by Rhodia and long chain betaines such as the product marketed under the trade name “Empigen BB” by Huntsman Corporation, and mixtures thereof.

Suitable nonionic surfactants include polyethoxylated sorbitol esters, especially poly-ethoxylated sorbitol monoesters, e.g., PEG sorbitan di-isostearate, and the products marketed under the trade name “Tween” by ICI; polycondensates of ethylene oxide and propylene oxide (polyoxamers), e.g., the products marketed under the trade name “Pluronic” by BASF; condensates of propylene glycol; polyethoxylated hydrogenated castor oil such as the products marketed under the trade name “Cremophors” by BASF; and sorbitan fatty esters marketed by ICI. Other effective nonionic surfactants include the polyalkyl(C₈-C₁₈) glucosides.

Suitable cationic surfactants include D,L-pyrrolidone-5-carboxylic acid salt of ethyl-cocoyl-L-arginate (CAE) marketed by Ajinomoto, and cocoamidopropyl (PTC), lauramidopropyl PG diammonium chloride phosphates and the like marketed by Uniqema. CAE and PTC have significant bioactivity and they therefore can be used as the cation of the binary cationic-anionic bioactive salts.

The choice of an effective surfactant system will differ somewhat for each bioactive salt of the invention. The choice of the surfactant system will depend upon the surfactant(s)' hydrophilic-lipophilic balance (HLB) to form a stable small particle micelle in an aqueous or aqueous-cosolvent medium.

Other adjuvants useful in formulating the bioactive salts of the invention in o/w or w/o type creams, gels, lotions and the like include: polyether-modified silicone, cyclic silicone, methyl polysilicone, polyoxyethylene castor oil, cetostearyl alcohol, neopentyl glycol dicaprate, sorbitan monostearate, polyvinyl alcohol, glycerin, “Carbox”, glyceryl ether, cholesteryl isostearate, ethanol, isopropanol, glycerol monostearate PEG 100 stearate, hydroxymethyl cellulose, cetyl alcohol, lauryl glucoside and the like.

### The Metathesis Reaction

As noted in the McGraw-Hill Dictionary of Scientific and Technical Terms (5^{th} Edition, 1994), metathesis is a reaction involving the exchange of elements or groups as in the general reaction:

AX+BY→AY+BX.

The metathesis reaction is straight forward and can be readily carried out in aqueous solutions using water alone or a mixture of water and up to about 85 wt. % of a solvent such as a C₁-C₄ alcohol, e.g., methanol, ethanol, isopropanol, n-butanol, etc. Typically the water alone or water-alcohol solvent will be utilized in an amount of about 40 to about 85 wt. %, based on the weight of the reaction mixture.

An alkali or alkaline earth metal (e.g., Na, K, Li, Ca, etc.) salt of the selected monomeric or polymeric anionic molecule is formed by reacting it with an equivalent amount of an alkali or alkaline earth metal hydroxide in water or water-alcohol solution. An acid salt, e.g., acetate, hydrohalide, gluconate, sulfate, etc. of the selected monomeric or polymeric cationic molecule is formed by reacting it with an equivalent amount of an acid such as acetic, hydrochloric, hydrobromic, gluconic acid, sulfuric, etc. in water or water-alcohol solution. Thereafter, an equivalent amount of the aqueous alkali or alkaline earth metal salt solution of the selected monomeric or polymeric anionic molecule is mixed with the aqueous acid salt solution of the selected monomeric or polymeric cationic molecule. The concentration of the reactants can vary from about 20 wt. % to about 60 wt. % of the total reaction mixture. Mixing is continued at room temperature for several minutes up to about one hour. The reaction product, i.e., the salt of the invention, may be readily recovered by decantation of the supernatant layer (which contains the byproduct salts) or by filtration. The solid layer consisting of the salt of the invention may be used as is for many of the materials recited above or dried (e.g., in air, in vacuo at a temperature of about 50 to about 130° C., etc.). If desired, the salt of the invention may be recrystallized using a solvent such that the solubility of the salt in the solvent is low at room temperature, but the solubility increases significantly near the boiling point of the solvent.

### The Acid-Base Reaction

It is preferred to use an acid-base reaction to prepare a salt of the invention if the selected monomeric or polymeric anionic molecule is capable of protonating the selected monomeric or polymeric cationic molecule in the form of its free base. The use of the acid-base reaction avoids the necessity of forming an alkali metal salt of the selected anionic molecule and the acid salt of the selected cationic molecule and having to dispose of the salt byproduct.

The acid-base reaction of a conjugate base (i.e., the free base) of the selected monomeric or polymeric cationic molecule with the conjugate acid (protonated) of the selected monomeric or polymeric anionic molecule may be illustrated by the following example:

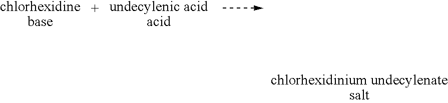

In order for the acid-base reaction to proceed, the acid component must have a transferable proton (Pₖₐ) to a basic (P_{kb}) molecule. The acid-base reaction is usually conducted in refluxing alcohol (e.g., a C₁-C₄ alcohol such as methanol, ethanol, isopropanol, n-butanol, etc.) or aqueous alcoholic solution (e.g., about 10 to about 90 wt. % water) and the reaction is typically complete in one hour or less. The salt may be readily recovered from the reaction mixture by filtration, air drying, removal of the solvent in vacuuo at a temperature of about 50 to about 130° C., etc. If desired, the salt may be recrystallized using a solvent such that the solubility of the salt in the solvent is low at room temperature, but the solubility increases significantly near the boiling point of the solvent.

### Applications of the Salts of the Invention

Set forth below is a representative list of some of the numerous possible applications of the salts of the present invention. It is to be understood that this list is presented for illustrative purposes only and should not be construed as representing any limitation as to possible applications of the salts. It is to be further understood that it is within the purview of the invention to combine the salts with conventional antioxidants, antibacterial agents, antifungal agents, hormones, vitamins, hydroxy acids, cleansers, soaps, shampoos, silicones, biocides, humectants, emollients, synthetic and/or natural oils, deodorizers, perfumes, colorants, preservatives, plant extracts and the like

Mammalian skin/hair/nail care products, e.g., sunscreens, suntan lotions, after-sun lotions, gels creams and sprays; antiperspirants; deodorants (liquids, powders, gels, roll-ons, sticks, sprays, pastes, creams, lotions); cleansing creams; skin conditioners; skin moisturizers; protectants; skin aging products; skin wrinkle-reduction products; acne treatment products; rosacea treatment products; age-spot reduction products; stretch-mark reduction products; pimple treatment products; skin soothing products; skin infection and lesion treatment products; skin-redness reduction products; varicose- and spider-vein reduction products; lotions; oils; hand/body creams; shaving gels, foams and creams; body washes; liquid and solid soap products; blood microcirculation improvement products; cellulite-reduction products; body toning products; skin penetration enhancers; skin whitening products; cosmetics; shampoos; shower gels; bubble baths; hair treatment products, e.g., medicated shampoos, mousses, waxes, conditioners, styling agents, lotions, sprays, gels, dyes and tints, colorant and non-colorant rinses, detangling lotions, hair curling and hair straightening products, hair wave products, etc.; nail treatment products; hand (or mechanical) dishwashing products; hand sanitizers and disinfectants; lipsticks and lip balms; salves; collodion; impregnated patches and strips for skin treatment; skin surface implants; impregnated or coated diapers; and the like.

The following examples shall serve to illustrate the various embodiments of the invention. The examples are given solely for the purpose of illustration and are not to be construed as limitations of the present invention since many variations are possible without departing from the spirit and scope of the invention. Unless otherwise indicated, all parts and percentages are on a weight basis.

### EXAMPLE 1

This example pertains to a lotion formulation containing a chlorhexidine-salicylate salt.

| Ingredient | Weight % |
|---|---|
| Salt | 0.5 |
| “Tego Betaine ZF” | 1.5 |
| Propylene Glycol | 15.0 |
| Propylene Glycol Dicaprylate-Dicaprate | 8.0 |
| Hydroxyethyl Cellulose | 0.25 |
| PEG 40 Stearate | 2.50 |
| “Stearath-2” | 1.00 |
| Water | QS |

### EXAMPLE 2

This example pertains to an aqueous micro-emulsion containing a salt resulting from the reaction of N-lauroyl-L-arginine-ethyl ester hydrochloride salt with sodium lactate.

| Ingredient | Weight % |
|---|---|
| Salt | 1.00 |
| Ethanol | 55.00 |
| Hydroxypropyl Cellulose | 0.50 |
| “Tego Betaine ZF” | 2.00 |
| Propylene Glycol | 10.00 |
| Dye | 0.020 |
| Water | QS |

What is claimed is:

1. A dermatological composition comprising a salt of a monomeric or polymeric cation and a monomeric or polymeric anion, wherein the cation is selected from the group consisting of an amidine moieity, a guanidine moieity and a biguanide moieity, and the anion comprises a carboxylic acid moieity.

2. The composition of claim 1 wherein the salt is formed by a metathesis reaction.

3. The composition of claim 1 wherein the cation comprises a guanidine moieity.

4. The composition of claim 3 wherein the guanidine moieity comprises a dibasic amino acid guanidine moieity.

5. The composition of claim 4 wherein the dibasic amino acid guanidine moieity comprises an N^{α}-(C₁-C₂₂) alkanoyl di-basic amino acid (C₁-C₂₂) alkyl ester.

6. The composition of claim 5 wherein the ester comprises an ester selected from the group consisting of N^{α}-lauroyl-L-arginine ethyl ester, N^{α}-lauroyl-L-histidine ethyl ester and N^{α}-lauroyl-L-tryptophan ethyl ester.

7. The composition of claim 6 wherein the ester comprises N^{α}-lauroyl-L-arginine ethyl ester.

8. The composition of claim 1 wherein the cation comprises a biguanide moieity.

9. The composition of claim 8 wherein the biguanide moieity is derived from a biguanide molecule selected from the group consisting of chlorhexidine, hexetidine, alexidine and polyhexamethylene biguanide hydrochloride.

10. The composition of claim 1 wherein the carboxylic acid moieity contains a saturated or unsaturated functional group selected from the group consisting of aliphatic, aromatic and alicyclic groups.

11. The composition of claim 10 wherein the carboxylic acid moieity is selected from the group of a monobasic aliphatic carboxylic acid moieity, a monobasic aromatic carboxylic acid moieity and a monobasic alicyclic carboxylic acid moieity.

12. The composition of claim 11 wherein the monobasic aliphatic carboxylic acid moieity is derived from a carboxylic acid molecule selected from the group of lauric acid, palmitic acid, myristic acid, oleic acid, stearic acid, dehydroacetic acid and undecylenic acid.

13. The composition of claim 11 wherein the monobasic aliphatic carboxylic acid moieity contains a hydroxyl group or a ketone group.

14. The composition of claim 13 wherein the monobasic aliphatic carboxylic acid moieity is derived from an aliphatic alpha-hydroxy carboxylic acid molecule selected from the group consisting of glycolic acid, gluconic acid, glyceric acid and lactic acid.

15. The composition of claim 11 wherein the monobasic aromatic carboxylic acid moieity is derived from salicylic acid.

16. The composition of claim 1 wherein the carboxylic acid moieity is derived from a carboxylic acid molecule containing at least two carboxylic acid groups.

17. The composition of claim 14 wherein the carboxylic acid molecule is selected from the group consisting of citric acid, malic acid, tartaric acid and azelaic acid.

18. The composition of claim 1 wherein the salt has a maximum solubility in aqueous media of about 5 wt. %.

19. The composition of claim 16 wherein the salt has a maximum solubility in aqueous media of 2 wt. %.

20. The composition of claim 1 wherein the salt is selected from the group consisting of the laurate of N^{α}-lauroyl-L-arginine ethyl ester, the salicylate of N^{α}-lauroyl-L-arginine ethyl ester, the lactate of N^{α}-lauroyl-L-arginine ethyl ester, the citrate of N^{α}-lauroyl-L-arginine ethyl ester, the malate of N^{α}-lauroyl-L-arginine ethyl ester, the gluconate of N^{α}-lauroyl-L-arginine ethyl ester, the azelate of N^{α}-lauroyl-L-arginine ethyl ester, the glycolate of N^{α}-lauroyl-L-arginine ethyl ester, the glycerate of N^{α}-lauroyl-L-arginine ethyl ester, the hyaluronate of N^{α}-lauroyl-L-arginine ethyl ester, the arachidonate of N^{α}-lauroyl-L-arginine ethyl ester, the oleate of N^{α}-lauroyl-L-arginine ethyl ester (C₁₈, unsaturated), the linoleate of N^{α}-lauroyl-L-arginine ethyl ester (C₁₈, polyunsaturated), the α-linoleate of N^{α}-lauroyl-L-arginine ethyl ester acid (ALA), the eicosapentaenoate of N^{α}-lauroyl-L-arginine ethyl ester acid (EPA), the docosahexaeonate of N^{α}-lauroyl-L-arginine ethyl ester (DHA), the erucate of N^{α}-lauroyl-L-arginine ethyl ester, the tartrate of N^{α}-lauroyl-L-arginine ethyl ester and the 3-hydroxypropionate of N^{α}-lauroyl-L-arginine ethyl ester.
